(19) United States
(12) Patent Application Publication
(10) Pub. No.: US 2004/0077607 A1
(43) Pub. Date: Apr. 22, 2004

(54) Aryl phosphate derivatives of d4T with potent anti-viral activity against hemorrhagic fever viruses

(76) Inventor: Fatih M. Uckun, White Bear Lake, MN (US)

Correspondence Address: MERCHANT & GOULD PC, P.O. BOX 2903, MINNEAPOLIS, MN, 55402-0903, US

(21) Appl. No.: 10/281,402

(22) Filed: Oct. 25, 2002

Related U.S. Application Data
(60) Provisional application No. 60/420,242, filed on Oct. 21, 2002.

(51) Int. Cl. A61K 31/675

(52) U.S. Cl. 514/85

(57) ABSTRACT

Methods for treating hemorrhagic fever viral infections by administering an aryl phosphate derivative of d4T having an electron-withdrawing substituent on the aryl group and an amino acid substituent on the phosphate group are described. A preferred aryl phosphate derivative of d4T is d4T-5′-[p-bromophenyl methoxyalaninyl phosphate].

### PRIORITY OF THE APPLICATION

This application claims priority to U.S. Provisional Application 60/______ entitled “ARYL PHOSPHATE DERIVATIVES OF d4T WITH POTENT ANTI-VIRAL ACTIVITY AGAINST HEMORRHAGIC FEVER VIRUSES” filed on Oct. 21, 2002.

### BACKGROUND OF THE INVENTION

The potential use of microorganisms as offensive agents is a growing concern for several reasons, including ease of production and dispersion, delayed onset, ability to cause high rates of morbidity and mortality, difficulties in rapid diagnosis, and very limited treatment options. Biological agents that have been identified as posing the greatest threats include variola major (smallpox), Bacillus anthracis (anthrax), Yersinia pestis (plague), Clostridium botulinum toxin (botulism), Francisella tularensis (tularaemia), and hemorrhagic fever viruses (Broussard LA, Mol Diagn 2001 Dec.;6(4):323-33).

Viral hemorrhagic fevers (VHF) are virus-induced, potentially fatal, acute febrile, hemorrhagic diseases reported from wide areas of the world. Hemorrhagic fever (HF) viruses are encapsulated, single-stranded RNA viruses that are associated with insect or rodent vectors whose interaction with humans defines the mode of disease transmission (Chen et al., Blood Coag. 2000. 11: 461-483). There are 14 HF viruses, which belong to four viral families: Arenaviridae, Bunyaviridae, Filoviridae, and Flaviviridae (Chen et al., 2000, Supra).

Arenaviruses are single-stranded RNA viruses and show a predilection for rodents as virus reservoirs (Enria et al., Arenaviruses In: Guerrant R L, Walker D H, Weller P F, eds. Tropical Infectious diseases: Principles, Pathogens, and Practice. Philadelphia: Churchill Livingstone; 1999: Chapter 111.). Pathogenic arenaviruses have been identified as the causative agents in Argentinian HF (virus: Junin), Bolivian HF (virus: Machupo), Venezuelan HF (virus: Guanarito), Brazilian HF (virus: Sabia), lymphocytic choriomeningitis (LCM) (virus: LCM), and Lassa fever (virus: Lassa) (Enria et al., 1999, Supra). Arenaviruses have a genome consisting of two single-strand RNA molecules, designated L and S, that contain essentially nonoverlapping sequence information and are ambisense in their coding arrangement (Fields et al., Fundamental Virology (3d. ed.) Lippincott-Raven 1996: 675-679). The general organization of the viral genome is well preserved across the virus family. The S segment encodes the major structural components including the internal nucleo-protein, NP, and the two external glycoproteins, GP-1 and GP-2. The L segment encodes the viral RNA-dependent RNA polymerase, and a potential structural and/or regulatory protein Z. The coding capacity of the two RNA molecules is limited to four defined open reading frames that yield five mature proteins.

Lassa fever is an acute viral disease found in every country of West Africa from Nigeria to Senegal that causes considerable morbidity and mortality (Frame et al., Am J Trop Med Hyg. 1970;19:670.). Lassa fever has an insidious onset, is initially difficult to diagnose, has “nonspecific” clinical symptoms which have been confused with yellow fever and typhoid, shows evidence of persistent infection, is tremendously contagious, and has a high mortality rate. Severe multi-organ involvement occurs in 5-10% of infections and case-fatality rates for hospitalized patients range from 15 to 25% (McCornick et al., Am J Trop Med Hyg. 1986;53:401.). Lassa fever has also been shown to be the cause of premature births and spontaneous abortions in pregnant women. The virus is transmitted by the respiratory route and by direct contact with contaminated materials. Lassa fever has emerged as a worldwide concern among public health officials, because its unique ability to spread from person to person, the risk of its importation by international travel, and renewed threats about the potential offensive use of HF viruses.

Currently, there are no antiviral drugs approved by the US Food and Drug Administration for treatment of HFVs. Small trials have shown that ribavirin may reduce mortality after infection with Lassa fever (McCormick et al., N. Engl. J Med. 1986;314:20-26). Other arenaviruses, such as Bolivian hemorrhagic fever (Machupo) (Kilgore et al., Clin. Infect. Dis. 1997;24:718-722) and Argentine hemorrhagic fever (Junin) (Enria et al., Antiviral Res. 1994;23:23-31) have also been successfully treated with ribavirin on a limited basis.

Ribavirin, (1-β-D-ribofuranosyl-1,2,4-triazole-3-carboxamide), is a broad spectrum antiviral guanosine analog (Huggins et al., Rev. of Infect. Dis., Vol. 11, Supp. 4, May-Jun. 1989). Ribavirin functions primarily as an IMP dehydrogenase inhibitor (Andrei et al., Antiviral Research, 22 (1993)45-75). However, the precise mechanism of action of ribavirin remains unknown (Cameron et al., Curr. Opin. Infect Dis. 2001;14(6):757-764). Accordingly, there is a need to identify anti-viral agents for effective treatment of HFV.

2′,3′-didehydro-2′,3′-dideoxythymidine (hereinafter “d4T”), is known as an inhibitor of the reverse transcriptase (RT) activity of the human immune deficiency virus (HIV). The bioactive form of this inhibitor, d4T-triphosphate, is generated intracellularly by the action of nucleoside kinase and nucleotide kinase. The rate-limiting step for the intracellular generation of the bioactive d4T metabolite d4T-triphosphate was reported to be the conversion of the nucleoside to its monophosphate derivative. (Balzarini et al., 1989, J.Biol. Chem. 264:6127; McGuigan et al., 1996, J. Med. Chem. 39:1748). Such compounds undergo intracellular hydrolysis to yield monophosphate derivatives that are further phosphorylated by thymidylate kinase to give the bioactive triphosphate derivatives in a thymidine kinase (TK)-independent fashion. U.S. Pat. No. 6,030,957 (Uckun et al.) disclosed that substitution of the aryl moiety of an aryl phosphate derivative of d4T, for example with halogen, enhances the ability of the compounds to undergo hydrolysis. One such compound is (d4T-5′-[p-bromophenyl methoxyalaninyl phosphate]). As described below, such substituted aryl phosphate derivatives of d4T have now been found to effectively inhibit HFV.

### SUMMARY OF THE INVENTION

The invention is provides a method for treating hemorrhagic fever by administering aryl phosphate derivatives of 2′,3′-didehydro-2′,3′-dideoxythymidine. Derivatives of d4T having the structure of Formula I exhibit antiviral activity against hemorrhagic fever viruses and are useful for treating hemorrhagic fever virus infections.

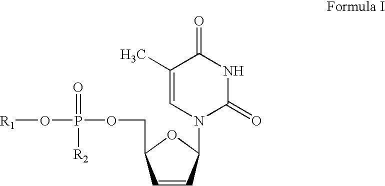

R₁ is an aryl group substituted with an electron-withdrawing group and R₂ is an amino acid or an ester of an amino acid.

In one embodiment of the invention, in the compound of Formula I, R₁ is phenyl substituted with an electron-withdrawing group such as halogen, and R₂ is an ester of an a-amino acid. Preferably, the compound of Formula I is d4T-5′-[p-bromophenyl methoxyalaninyl phosphate] where R₁ is a phenyl group substituted with bromine or chlorine at the para position and R₂ is the methyl ester of alanine.

The oral or intravenous administration of substituted aryl phosphate derivatives such as d4T-5′-[p-bromophenyl methoxyalaninyl phosphate] results in the formation of two key metabolites: alaninyl-d4T-monophosphate (Ala-d4T-MP) and d4T. Administration of d4T-5′-[p-bromophenyl methoxyalaninyl phosphate] results in more prolonged systemic exposure to Ala-d4T-MP as well as d4T than administration of an equimolar dose of either metabolite. As shown in the Examples below, each metabolite has a significantly longer elimination half-life when formed from the administration of d4T-5′-[p-bromophenyl methoxyalaninyl phosphate] than when administered directly.

The invention provides a method for treating hemorrhagic fever virus infections comprising administering an effective amount of a compound of Formula IV:

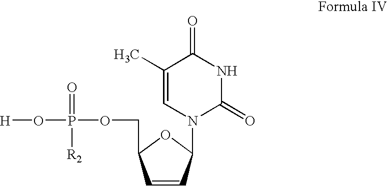

where R₂ is an amino acid or amino acid ester residue, such as the methyl ester of alanine.

### BRIEF DESCRIPTION OF THE DRAWINGS

FIG. 1 shows representative HPLC chromatograms of blank plasma (A), blank plasma spiked with d4T-5′-[p-bromophenyl methoxyalaninyl phosphate], d4T and Ala-d4T-MP (B), and plasma samples 10 minutes after intravenous injection of 100 mg/kg d4T-5′-[p-bromophenyl methoxyalaninyl phosphate] (C).

FIG. 2 is a plot showing the stability of d4T-5′-[p-bromophenyl methoxyalaninyl phosphate] in plasma (A) and in whole blood (B) as a function of time.

FIG. 3 is a plot showing the stability of d4T-5′-[p-bromophenyl methoxyalaninyl phosphate] and the formation of key metabolites in plasma in presence of paraozon (A), physostigmine (B), and EDTA (C) as a function of time.

FIG. 4 is a plot showing the stability of d4T-5′-[p-bromophenyl methoxyalaninyl phosphate] and the formation of key metabolites in liver homogenate as a function of time.

FIG. 5 is a plot showing the stability of d4T-5′-[p-bromophenyl methoxyalaninyl phosphate] and formation of key metabolites in gastric fluid (A) and in intestinal fluid (B) as a function of time.

FIG. 6 shows plots of the plasma concentrations of total d4T-5′-[p-bromophenyl methoxyalaninyl phosphate] (A), d4T-5′-[p-bromophenyl methoxyalaninyl phosphate]-A (B) and d4T-5′-[p-bromophenyl methoxyalaninyl phosphate]-B (C) as a function of time in Balb/C mice following intravenous injection of d4T-5′-[p-bromophenyl methoxyalaninyl phosphate] (100 mg/kg, 4 mice per time-point).

FIG. 7 shows (A) the pharmacokinetic model for describing d4T-5′-[p-bromophenyl methoxyalaninyl phosphate], Ala-d4T-MP and d4T after intravenous injection of d4T-5′-[p-bromophenyl methoxyalaninyl phosphate]; and (B) the plasma concentrations of d4T-5′-[p-bromophenyl methoxyalaninyl phosphate], Ala-d4T-MP and d4T as a function of time in Balb/C mice following intravenous injection of d4T-5′-[p-bromophenyl methoxyalaninyl phosphate] (100 mg/kg, 4 mice per time-point).

FIG. 8 shows (A) a pharmacokinetic model for describing Ala-d4T-MP and d4T after intravenous injection of Ala-d4T-MP; and (B) plasma concentrations of Ala-d4T-MP and d4T as a function of time in Balb/C mice following intravenous injection of Ala-d4T-MP (75 mg/kg, 5 mice per time-point).

FIG. 9 shows plasma concentration of d4T as a function of time in BALB/C mice following intravenous injection of D4T (40 mg/kg, 5 mice per time-point).

FIG. 10 shows plasma concentrations of Ala-d4T-MP (A) and d4T (B) as a function of time in BALB/C mice following oral administration of d4T-5′-[p-bromophenyl methoxyalaninyl phosphate] (100 mg/kg, 4 mice per time-point).

FIG. 11 shows survival percent (%) of CBA mice as a function of time after inoculation with Josiach strain of Lassa virus and treatment with one of: vehicle, d4T-5′-[p-bromophenyl methoxyalaninyl phosphate] (25 mg/kg), or d4T-5′-[p-bromophenyl methoxyalaninyl phosphate] (50 mg/kg).

### DETAILED DESCRIPTION OF THE INVENTION

As used herein, the following terms and phrases have the indicated definitions:

The term “administering” refers to providing to a mammal in any manner including: orally, parentally (including subcutaneous injection, intravenous, intramuscular, intrasternal or infusion techniques), by inhalation spray, topically, by absorption through a mucous membrane, or rectally, in dosage unit formulations containing conventional non-toxic pharmaceutically acceptable carriers, adjuvants or vehicles, and other known modes of drug delivery.

The term “amino acid” refers to any of the naturally occurring amino acids, as well as their opposite enantiomers or racemic mixture of both enantiomers, synthetic analogs, and derivatives thereof. The term includes, for example, α-, β-, γ-, δ-, and ω-amino acids. Suitable naturally occurring amino acids include glycine, alanine, valine, leucine, isoleucine, proline, threonine, serine, methionine, cysteine, aspartic acid, asparagine, glutamic acid, glutamine, arginine, lysine, phenylalanine, tryptophan, tyrosine, and histidine. Synthetic, or unnatural, amino acids such as, for example, trifluoroleucine, p-fluorophenylalanine, and 3-triethylalanine can be used. The term amino acid includes esters of the amino acids. Esters include lower alkyl esters in which the alkyl group has one to seven carbon atoms, preferably one to four carbon atoms such as, for example, methyl, ethyl, propyl, and butyl. The amino group of the amino acid or ester thereof is attached to the phosphate group in Formula I.

The term “animal” includes, but is not limited to mammals, such as humans.

The term “aryl” includes aromatic groups such as, for example, phenyl, naphthyl, and anthryl.

The term “electron-withdrawing groups” includes groups such as halo (—NO₂, —CN, —SO₃H, —COOH, —CHO, —COR (where R is a (C₁ to C₄) alkyl), and the like.

The term “halo” or “halogen” is used to describe an atom selected from the group of Bromine (Br), Chlorine (Cl), Fluorine (F) and Iodine (I).

The term “protecting” or “preventing” refers to taking advance measures against a possible or probable infection to prevent the morbidity and mortality normally associated with a disease-causing agent.

The term “viral hemorrhagic fever infection” refers to those infections caused by encapsulated, single-stranded RNA viruses that may cause potentially fatal acute febrile, hemorrhagic disease, including those viruses that belong to the four viral families: Arenaviridae, Bunyaviridae, Filoviridae, and Flaviviridae. A specific example is Lassa Virus (Borio et al., 2002 JAMA 287(18) 2391-2405).

The term “treating” refers to caring for or dealing with a condition medically and may include alleviating symptoms, eliminating infection, impeding infection, or otherwise improving health. Symptoms of hemorrhagic fever virus infection typically include fever, hypotension, relative bradycardia, tachypnea, conjunctivitis, and pharyngitis. Most types of infections with a hemorrhagic fever virus are also associated with cutaneous flushing or a skin rash. Later symptoms include signs of progressive hemorrhagic diathesis, such as petechiae, mucous membrane and conjunctival hemorrhage; hematuria; hematemesis; and melena, as well as disseminated intravascular coagulation and circulatory shock. Central nervous system dysfunction may be present and manifested by delirium, convulsions, cerebellar signs, or coma. (Borio et al., 2002 JAMA 287(18) 2391-2405.)

Compounds Useful in the Method Invention

The invention is directed to methods of using aryl phosphate derivatives of 2′,3′-didehydro-2′,3′-dideoxythymidine (derivatives of d4T) to treat or inhibit hemorrhagic fever viral infections. More particularly, the present invention provides methods for treating or inhibiting hemorrhagic fever viral infections in a mammal by administering an aryl phosphate derivative of d4T having an electron-withdrawing substituent on the aryl group and an amino acid substituent on the phosphate group as in Formula I:

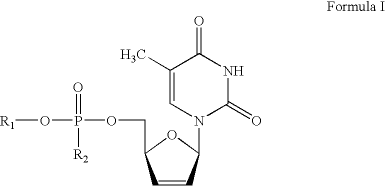

where R₁ is an aryl group substituted with an electron-withdrawing group and R₂ is an amino acid or an ester of an amino acid.

The compounds of Formula I can also be in the form of pharmaceutically acceptable salts. Pharmaceutically acceptable salts are formed with organic and inorganic acids. Examples of suitable acids for salt formation with the amino group of the amino acid or amino acid ester residue of a compound of Formula I include, but are not limited to hydrochloric, sulfuric, phosphoric, acetic, citric, oxalic, malonic, salicylic, malic, gluconic, fumaric, succinic, asorbic, maleic, methanesulfonic, and the like. The salts are prepared by contacting the free base form with a sufficient amount of the desired acid to produce either a mono or di, etc. salt in the conventional manner. Suitable bases for the formation of a salt with the carboxylate group of the amino acid residue of a compound of Formula I include, for example, sodium hydroxide, sodium carbonate, sodium bicarbonate, potassium hydroxide, potassium carbonate, and potassium bicarbonate.

In one embodiment of Formula I, R₁ is a phenyl group substituted with an electron-withdrawing group and R₂ is an α-amino acid or ester thereof as shown in Formula II:

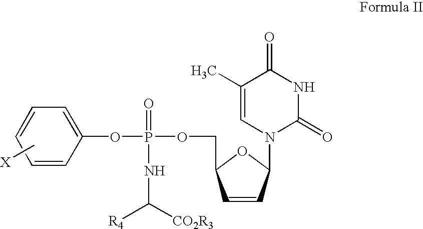

In Formula II, X is an electron-withdrawing group such as halo —NO₂, —CN, —SO₃H, —COOH, —CHO, —COR (where R is a (C₁ to C₄) alkyl), and the like. R₃ is hydrogen or an alkyl of one to seven carbon atoms, preferably an alkyl of one to four carbon atoms, such as, for example, methyl, ethyl, propyl, and butyl. R₄ is hydrogen (e.g., as in glycine), an alkyl (e.g. as in alanine, valine, leucine, isoleucine, proline), a substituted alkyl (e.g., as in threonine, serine, methionine, cysteine, aspartic acid, asparagine, glutamic acid, glutamine, argine, and lysine), an arylalkyl (e.g., as in phenylalanine and tryptophan), a substituted arylalkyl (e.g., as in tyrosine), or a heteroalkyl (e.g., as in histidine).

One embodiment, the compound of Formula II is d4T-5′-[p-bromophenyl methoxyalaninyl phosphate], (d4T-5′-[p-bromophenyl methoxyalaninyl phosphate]) where X is bromo attached to the phenyl group in the para position, R₄ is methyl, and R₃ is methyl. The structure of d4T-5′-[p-bromophenyl methoxyalaninyl phosphate] is shown in Formula III:

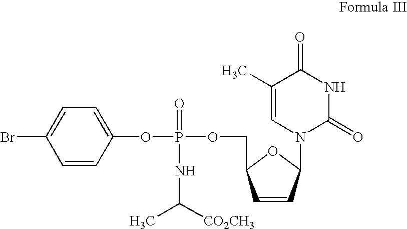

Pharmacokinetics

Previous in vitro studies have shown that an electron-withdrawing group at the para position of the phenyl group enhances the rate of hydrolysis and thereby enhances production of a key metabolite alaninyl-d4T-monophosphate (Ala-d4T-MP) relative to the unsubstituted aryl phosphate derivative (Venkatachalam et al., Bioorg. Med. Chem. Lett., 8, 3121 (1998); Vig et al., Antiviral Chem. Chemother., 9, 445 (1998); and U.S. Pat. No. 6,030,957 (Uckun et al.)).

The anti-viral agent d4T-5′-[p-bromophenyl methoxyalaninyl phosphate] (referred to in Scheme 1 below as HI-113) is quickly metabolized in vivo to form two metabolites: 2′,3′-didehydro-3′-deoxythymidine (d4T) and alaninyl-d4T-monophosphate (Ala-d4T-MP) as shown in Scheme 1. Ala-d4T-MP can also be metabolized further to yield d4T. The metabolite d4T had not been found in earlier in vitro studies with cells.

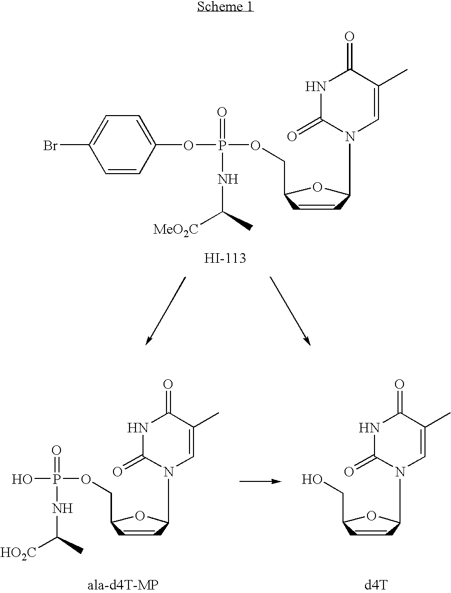

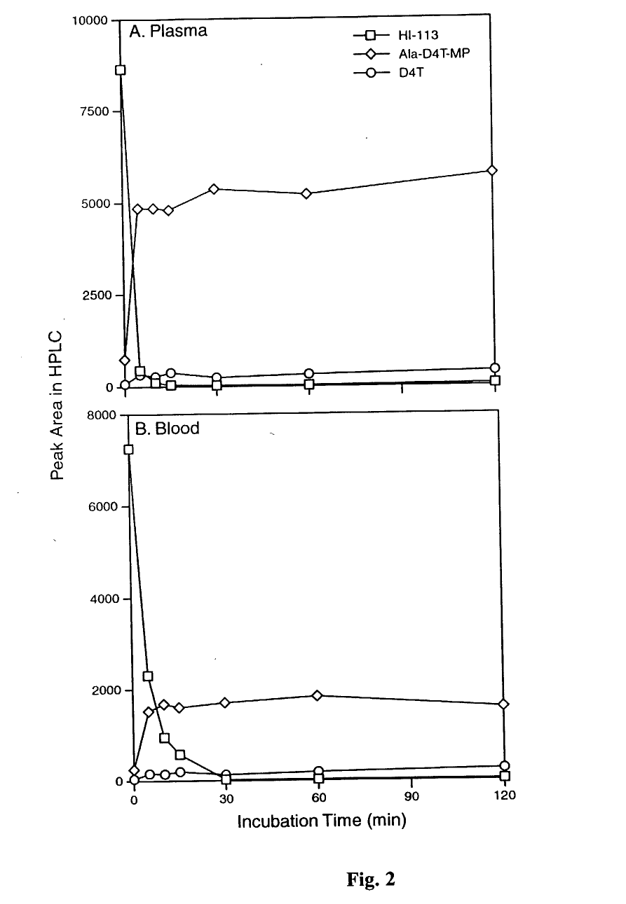

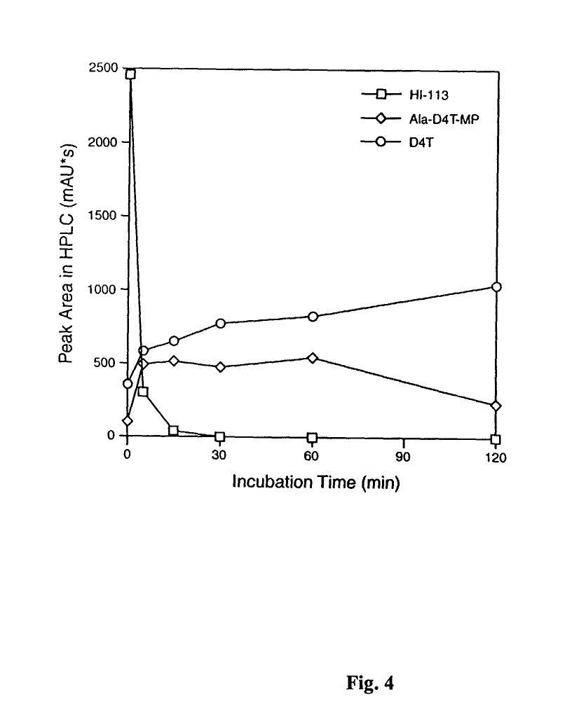

d4T-5′-[p-bromophenyl methoxyalaninyl phosphate] readily metabolizes in either plasma or whole blood to form Ala-d4T-MP and a small amount of d4T (see FIG. 2). Ala-d4T-MP is stable both in plasma and in whole blood. These results indicate that other enzymes (e.g., liver enzymes) are needed to form d4T by hydrolysis of either Ala-d4T-MP or d4T-5′-[p-bromophenyl methoxyalaninyl phosphate]. This hypothesis is consistent with the formation of a significant amount of d4T after incubation of d4T-5′-[p-bromophenyl methoxyalaninyl phosphate] with a liver homogenate (see FIG. 4).

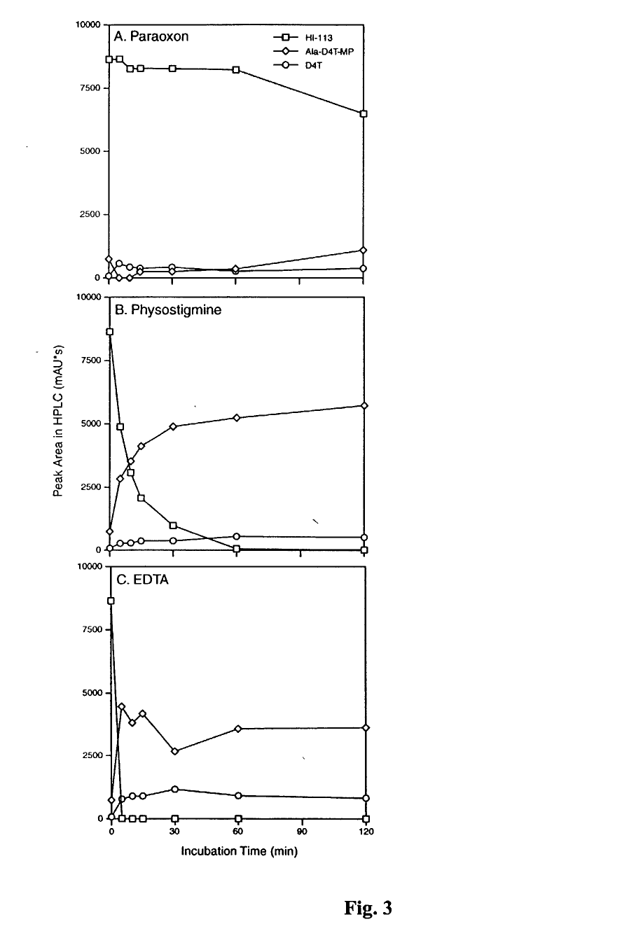

Paraoxon, an inhibitor of both cholinesterase and carboxylesterase (Augustinsson, Ann. N. Y. Acad. Sci., 94, 884 (1961); McCracken et al., Biochem. Pharmacol., 46, 1125 (1993); Madhu et al., J. Pharm. Sci., 86, 971 (1997)), significantly prevented the hydrolysis of d4T-5′-[p-bromophenyl methoxyalaninyl phosphate] to Ala-d4T -MP and d4T, suggesting that both cholinesterase and carboxylesterase are important for metabolism of d4T-5′-[p-bromophenyl methoxyalaninyl phosphate] (see FIG. 3A). Physostigmine, an inhibitor of cholinesterase, partially prevented the hydrolysis of d4T-5′-[p-bromophenyl methoxyalaninyl phosphate], which further supports the importance of cholinesterase in hydrolysis of d4T-5′-[p-bromophenyl methoxyalaninyl phosphate] (see FIG. 3B). EDTA, an inhibitor of arylesterase, did not affect the hydrolysis of d4T-5′-[p-bromophenyl methoxyalaninyl phosphate], indicating that arylesterase is probably not involved in the hydrolysis of d4T-5′-[p-bromophenyl methoxyalaninyl phosphate] (see FIG. 3C).

Elimination Half-Life

The elimination half-life of intravenously administered d4T is fairly similar to the elimination half-life of d4T formed after intravenous administration of Ala-d4T-MP (t_{1/2} of 30.3 minutes vs. 34.0 minutes) as shown in the Examples below. In contrast, the elimination half-life for d4T formed after intravenous administration of d4T-5′-[p-bromophenyl methoxyalaninyl phosphate] was significantly prolonged (t_{1/2} of 114.8 minutes). Similarly, the elimination half-life for Ala-d4T-MP formed from d4T-5′-[p-bromophenyl methoxyalaninyl phosphate] was significantly longer than the t_{1/2} for Ala-d4T-MP administered intravenously (t_{1/2} of 129.2 minutes vs. 28.5 minutes). The intravenous administration of d4T-5′-[p-bromophenyl methoxyalaninyl phosphate] results in prolonged systemic exposure to both Ala-d4T-MP and d4T compared to administration of equimolar dose of Ala-d4T-MP or d4T due to apparently longer elimination half-lives of d4T-5′-[p-bromophenyl methoxyalaninyl phosphate]-derived metabolites.

Following intravenous administration, the elimination half-life (t_{1/2}) of d4T-5′-[p-bromophenyl methoxyalaninyl phosphate] was 3.5 minutes with a systemic clearance (CL) of 160.9 ml/min/kg. Different estimates for systemic clearance (CL) values were obtained for the two diastereomers of d4T-5′-[p-bromophenyl methoxyalaninyl phosphate] (d4T-5′-[p-bromophenyl methoxyalaninyl phosphate]A is 208.2 ml/min/kg and d4T-5′-[p-bromophenyl methoxyalaninyl phosphate]B is 123.9 ml/min/kg), but both were completely metabolized within 30 minutes. d4T-5′-[p-bromophenyl methoxyalaninyl phosphate] was converted to the active metabolites Ala-d4T-MP (23%) and d4T (24%). The tₘₐₓ values for Ala-d4T-MP and d4T formed from intravenously administered d4T-5′-[p-bromophenyl methoxyalaninyl phosphate] were 5.9 minutes and 18.7 minutes, respectively.

Intravenous administration of Ala-d4T-MP results in formation of d4T (15%). Ala-d4T-MP can also be used as a d4T prodrug. The invention provides a method for treating hemorrhagic fever virus infections by administering an effective amount of a compound of Formula IV:

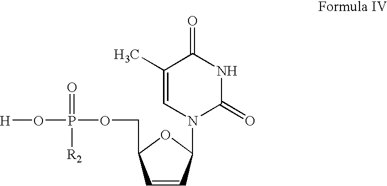

where R₂ is an amino acid or esterified thereof.

Salts

The compounds of Formula I to IV can also be in the form of pharmaceutically acceptable salts. Pharmaceutically acceptable salts can be formed with organic and inorganic acids. Examples of suitable acids for salt formation with the amino group of the amino acid or amino acid ester residue of Formula IV include, but are not limited to, hydrochloric, sulfuric, phosphoric, acetic, citric, oxalic, malonic, salicylic, malic, gluconic, fumaric, succinic, asorbic, maleic, methanesulfonic, and the like. The salts can be prepared by contacting the free base form with a sufficient amount of the desired acid to produce either a mono or di, etc. salt in the conventional manner. Suitable bases for the formation of a salt with the carboxylate group of the amino acid residue of Formula IV include, for example, sodium hydroxide, sodium carbonate, sodium bicarbonate, potassium hydroxide, potassium carbonate, and potassium bicarbonate.

Bioavailability

Orally administered d4T-5′-[p-bromophenyl methoxyalaninyl phosphate] also yielded Ala-d4T-MP and d4T as the major metabolites. No parent d4T-5′-[p-bromophenyl methoxyalaninyl phosphate] was detectable in the blood after oral administration. Although d4T-5′-[p-bromophenyl methoxyalaninyl phosphate] is stable in gastric fluid and can be absorbed in the stomach, it can quickly hydrolyze in blood. On the other hand, d4T-5′-[p-bromophenyl methoxyalaninyl phosphate] decomposes readily in intestinal fluid to form Ala-d4T-MP. This metabolite can be absorbed in the intestine and then further metabolized to yield d4T in the blood. The tₘₐₓ and t_{1/2} values for d4T in mice were longer when derived from orally administered d4T-5′-[p-bromophenyl methoxyalaninyl phosphate] (42.4 minutes and 99.0 minutes, respectively) than from orally administered d4T (5 minutes and 18 minutes, respectively). The tₘₐₓ value is higher but the t_{1/2} value is lower for orally administered d4T-5′-[p-bromophenyl methoxyalaninyl phosphate] compared to intravenously administered d4T-5′-[p-bromophenyl methoxyalaninyl phosphate]. The estimated bioavailabilities of Ala-d4T-MP and d4T were approximately 12% and 48%, respectively, after oral administration of d4T-5′-[p-bromophenyl methoxyalaninyl phosphate]. However, the bioavailability of d4T metabolized from d4T-5′-[p-bromophenyl methoxyalaninyl phosphate] (48%) was lower than that of orally administered D4T (98%).

The in vivo pharmacokinetics, metabolism, toxicity, and antiretroviral activity of d4T-5′-[p-bromophenyl methoxyalaninyl phosphate] in rodent species has been investigated (Uckun et al., Arzneimittelforschung/Drug Research, 2002, (in press)). In mice and rats, d4T-5′-[p-bromophenyl methoxyalaninyl phosphate] was very well tolerated without any detectable acute or subacute toxicity at single intraperitoneal or oral bolus dose levels as high as 500 mg/kg (Uckun et al., 2002, (Supra)). Notably, daily administration of d4T-5′-[p-bromophenyl methoxyalaninyl phosphate] intraperitoneally or orally for up to 8 consecutive weeks was not associated with any detectable toxicity in mice or rats at cumulative dose levels as high as 6.4 g/kg (Uckun et al., 2002, (Supra)). In accordance with its safety profile in rodent species, a four-week d4T-5′-[p-bromophenyl methoxyalaninyl phosphate] treatment course with twice daily administration of hard gelatin capsules containing 25 mg/kg -100 mg/kg d4T-5′-[p-bromophenyl methoxyalaninyl phosphate] was very well tolerated by dogs and cats at cumulative dose levels as high as 8.4 g/kg (Uckun et al., Antimicrob. Agents Chemother. (submitted 2002)).

Administration Methods

Compounds of Formulas I to IV can be formulated as pharmaceutical compositions and administered to a mammalian host, including a human patient in a variety of forms adapted to the chosen route of administration. The compounds are typically administered in combination with a pharmaceutically acceptable carrier, and can be combined with specific delivery agents, including targeting antibodies or cytokines.

Useful Dose

When used in vivo to inhibit hemorrhagic fever virus, the administered dose is that effective to have the desired effect, such as sufficient to reduce or eliminate one or more symptom of hemorrhagic fever. Appropriate amounts can be determined by those skilled in the art, extrapolating using known methods and relationships, from the in vivo animal model data provided in the Specification and Examples.

In general, the dose of the aryl phosphate derivatives of d4T effective to achieve therapeutic treatment of HFV infection, including reduction or prevention of symptoms or effects of HFV infection such as increased survival time, is in the approximate range of about 1-500 mg/kg body weight/dose, preferably about 10-100 mg/kg body weight/dose, and approximately 800-1000 mg/kg body weight per week of a cumulative dose.

The effective dose to be administered will vary with conditions specific to each patient. In general, factors such as the viral burden, host age, metabolism, sickness, prior exposure to drugs, and the like, contribute to the expected effectiveness of a drug. One skilled in the art will use standard procedures and patient analysis to calculate the appropriate dose, extrapolating from the data provided in the Examples. In general, a dose which delivers about 1-100 mg/kg body weight is expected to be effective, although more or less may be useful.

In addition, the compositions of the invention may be administered in combination with other therapies. In such combination therapy, the administered dose of the compounds may be less than for single drug therapy.

The compounds can be administered orally, parentally (including subcutaneous injection, intravenous, intramuscular, intrasternal or infusion techniques), by inhalation spray, topically, by absorption through a mucous membrane, or rectally, in dosage unit formulations containing conventional non-toxic pharmaceutically acceptable carriers, adjuvants or vehicles. Pharmaceutical compositions of the invention can be in the form of suspensions or tablets suitable for oral administration, nasal sprays, creams, and sterile injectable preparations, such as sterile injectable aqueous or oleagenous suspensions or suppositories.

For oral administration as a suspension, the compositions can be prepared according to techniques well known in the art of pharmaceutical formulation. The compositions can contain microcrystalline cellulose for imparting bulk, alginic acid or sodium alginate as a suspending agent, methylcellulose as a viscosity enhancer, and sweeteners or flavoring agents. As immediate release tablets, the compositions can contain microcrystalline cellulose, starch, magnesium stearate and lactose or other excipients, binders, extenders, disintegrants, diluents and lubricants known in the art.

For administration by inhalation or aerosol, the compositions can be prepared according to techniques well known in the art of pharmaceutical formulation. The compositions can be prepared as solutions in saline, using benzyl alcohol or other suitable preservatives, absorption promoters to enhance bioavailability, fluorocarbons or other solubilizing or dispersing agents generally known in the art.

For administration as injectable solutions or suspensions, the compositions can be formulated according to techniques well known in the art, using suitable dispersing or wetting and suspending agents, such as sterile oils, including but not limited to, synthetic mono- or diglycerides, and fatty acids, including oleic acid.

For rectal administration as suppositories, the compositions can be prepared by known methods, for example, by mixing with a suitable non-irritating excipient, such as cocoa butter, synthetic glyceride esters or polyethylene glycols, that are solid at ambient temperatures, but liquefy or dissolve in the rectal cavity to release the drug.

Solutions or suspensions of the compounds can be prepared in water, isotonic saline (PBS), and the like, and optionally can be mixed with a nontoxic surfactant. Dispersions may also be prepared by known methods, for example in glycerol, liquid polyethylene, glycols, DNA, vegetable oils, triacetin, and mixtures thereof. Under ordinary conditions of storage and use, these preparations may contain a preservative, for example, to prevent the growth of microorganisms.

The pharmaceutical dosage form suitable for injection or infusion use can include sterile, aqueous solutions or dispersions, sterile powders comprising an active ingredient, and the like, that are adapted for the extemporaneous preparation of sterile injectable or infusible solutions or dispersions. In all cases, the ultimate dosage form is preferable be sterile, fluid, and stable under the conditions of manufacture and storage. The liquid carrier or vehicle can be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. The proper fluidity can be maintained, for example, by the formation of liposomes, by the maintenance of the required particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Prolonged absorption of the injectable compositions can be brought about by the inclusion in the composition of agents delaying absorption—for example, aluminum monosterate hydrogels and gelatin.

Sterile injectable solutions are prepared by incorporating the conjugates in the required amount in the appropriate solvent with various other ingredients as enumerated above and, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

### EXAMPLES

The synthetic procedures for the preparation of d4T-5′-[p-bromophenyl methoxyalaninyl phosphate], Ala-d4T-MP and d4T have been previously described in detail (Venkatachalam et al., Bioorg. Med. Chem. Lett., 8, 3121 (1998); Vig et al., Antiviral Chem. Chemother., 9, 445, (1998) the compounds of Formula I to III can also be synthesized as described in U.S. Pat. No. 6,030,957 (Uckun et al.)) which patent is incorporated herein by reference.

### Example 1

### Quantitative HPLC For Detection of d4T-5′-[p-bromophenyl methoxyalaninyl phosphate] and Its Metabolites

The HPLC system used for these studies was a Hewlett Packard (Palo Alto, Calif.) series 1100 instrument equipped with a quaternary pump, an autosampler, an automatic electronic degasser, an automatic thermostatic column compartment, a diode array detector and a computer with Chemstation software for data analysis (Chen et al., J. Chromatogr. B., 724, 157 (1999); Chen et al., J. Chromatogr. B., 727, 205 (1999); and Chen et al., J. Liq. Chromatogr., 22, 1771 (1999)). The analytical column used was a Zobax SB-Phenyl (5 μm, Hewlett Packard, Inc.) column attached to a guard column (Hewlett Packard, Inc.). The column was equilibrated prior to data collection. The linear gradient mobile phase (flow rate=1.0 mL/minute) was: 100% A/0% B at 0 minutes, 88% A/12% B at 20 minutes, 8% A/92% B at 30 minutes (A: 10 mM ammonium phosphate buffer, pH 3.7; B: acetonitrile). The detection wavelength was 268 nm, the peakwidth was less than 0.03 minutes, the response time was 0.5 seconds, and the slit was 4 nm.

HPLC-grade reagents and deionized, distilled water were used in this study. Acetonitrile was purchased from Burdick & Jackson (Allied Signal Inc., Muskegon, Mich.). Acetic acid was purchased from Fisher Chemicals (Fair Lawn, N.J.). Ammonium phosphate and phosphoric acid were purchased from Sigma-Aldrich (St. Louis, Mo.).

Plasma samples (200 μL) were mixed 1:4 with acetone (800 μL) and vortexed for at least 30 seconds. Following centrifugation, the supernatant was transferred into a clean tube and was dried under nitrogen. A 50 μL solution of 50% methanol in 200 mM HCl was used to reconstitute the extraction residue, and 40 μL was injected into the HPLC.

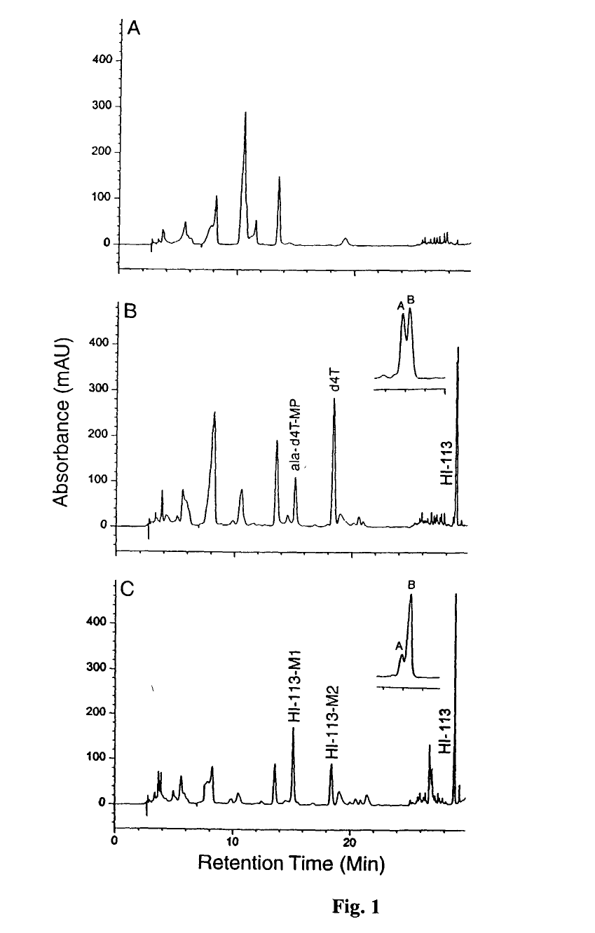

The chromatographic retention times (RT) measured for d4T-5′-[p-bromophenyl methoxyalaninyl phosphate] and its metabolites in spiked samples were 28.7±0.02 minutes (d4T-5′-[p-bromophenyl methoxyalaninyl phosphate]A; n=13; FIG. 1B), 28.9±0.02 minutes (d4T-5′-[p-bromophenyl methoxyalaninyl phosphate]B; n=13; FIG. 1C), 15.3±0.2 minutes (Ala-d4T-MP; n=30) and 18.5±0.1 minutes (d4T; n=30). d4T-5′-[p-bromophenyl methoxyalaninyl phosphate]A and d4T-5′-[p-bromophenyl methoxyalaninyl phosphate]B are diastereomers of d4T-5′-[p-bromophenyl methoxyalaninyl phosphate]. At these retention times, no significant interference peaks from the blank plasma were observed (FIGS. 1A and 1B).

The hydrochloric acid component of the reconstituted solutions played a role in the chromatography of d4T-5′-[p-bromophenyl methoxyalaninyl phosphate] and its metabolites; the acid protonated Ala-d4T-MP. No peak appeared in the chromatogram for this metabolite in the absence of hydrocholoric acid in the reconstituted solution. The acidic solution decreased the stability of Ala-d4T-MP, however. Therefore, all of the extracted samples were analyzed immediately after reconstitution.

### Example 2

### Stability of d4T-5′-[p-bromophenyl methoxyalaninyl phosphate] and Ala-d4T-MP in Whole Blood and Plasma

Whole blood and plasma samples were spiked with d4T-5′-[p-bromophenyl methoxyalaninyl phosphate] and Ala-d4T-MP to yield final concentrations of 250 μM d4T-5′-[p-bromophenyl methoxyalaninyl phosphate] and 100 μM Ala-d4T-MP, respectively. The whole blood samples were placed in a 37° C. water bath, while plasma samples were stored at −20° C. At a predetermined time, an aliquot (100 l) of spiked whole blood or plasma was extracted by adding 400 μl of acetone to induce precipitation of proteins, as described above. The absolute peak area was used to evaluate the stability of d4T-5′-[p-bromophenyl methoxyalaninyl phosphate] and Ala-d4T-MP.

The results shown in FIGS. 2A and 2B indicate that d4T-5′-[p-bromophenyl methoxyalaninyl phosphate] is very unstable in plasma and in whole blood. Following incubation with plasma, over 95% of the d4T-5′-[p-bromophenyl methoxyalaninyl phosphate] decomposed after 5 minutes. In the whole blood samples, 68%, 87%, and 92% of the d4T-5′-[p-bromophenyl methoxyalaninyl phosphate] decomposed in samples taken at 5, 10, and 15 minutes, respectively. In both the plasma and whole blood samples, decomposition of d4T-5′-[p-bromophenyl methoxyalaninyl phosphate] was complete within 30 minutes. (see Table 1 for plasma data). Thus, samples were extracted immediately after the samples were obtained. In contrast, Ala-d4T-MP was stable in both whole blood and plasma for 1 day.

### Example 3

### Stability of d4T-5′-[p-bromophenyl methoxyalaninyl phosphate] in Plasma in the Presence of Selective Esterase Inhibitors

Plasma samples were pre-incubated with the esterase inhibitors paraoxon (final concentration of 0.1 mM), physostigmine (final concentration of 0.1 mM), and EDTA (final concentration of 1M) at 37° C. for 30 minutes. Then d4T-5′-[p-bromophenyl methoxyalaninyl phosphate] was added to yield final concentrations of 250 μM. At a predetermined time, an aliquot (100 μl) of spiked plasma was extracted by adding 400 μl of acetone to induce precipitation of proteins, as described above. Decomposition of d4T-5′-[p-bromophenyl methoxyalaninyl phosphate] in plasma was significantly inhibited by paraoxon, partially inhibited by physostigmine, but not affected by EDTA (see FIGS. 3A, 3B, and 3C as well as Table 1). The data shown in Table 1 was calculated as mean percent hydrolysis from two experiments.

TABLE 1
Effect of Selective Esterase Inhibitors on Metabolism
of d4T-5′-[p-bromophenyl methoxyalaninyl
phosphate] in Plasma

| | | Paraoxon | | |
|---|---|---|---|---|
| | | (0.1 mM) | Physostigmine | EDTA |
| | No | cholinesterase & | (0.1 mM) | (1 mM) |
| Specificity | inhibitor | carboxylesterase | cholinesterase | arylesterase |
| 5 min | 95% | 0% | 43% | 99% |
| 10 min | 98% | 2% | 65% | 100% |
| 15 min | 99% | 2% | 76% | 100% |
| 30 min | 100% | 2% | 89% | 100% |
| 60 min | 100% | 3% | 100% | 100% |
| 120 min | 100% | 24% | 100% | 100% |

### Example 4

### Stability of d4T-5′-[p-bromophenyl methoxyalaninyl phosphate] in Murine Liver Homogenates

Fresh mouse liver was obtained from Balb/c mice and homogenated in 1×PBS (1:1, W/V) using a Polytron (PT-MR2000) homogenizer (Kinematical AG, Littau, Switzerland). d4T-5′-[p-bromophenyl methoxyalaninyl phosphate] was added to the liver homogenate to yield a final concentration of 100 μM. At a predetermined time, an aliquot (100 μl) of spiked liver homogenate was extracted by adding 400 μl of acetone to induce precipitation of proteins, as described above.

The compound d4T-5′-[p-bromophenyl methoxyalaninyl phosphate] decomposed after incubation with the liver homogenate within 30 minutes (FIG. 4), similar to the data obtained in plasma. However, unlike in plasma, significant amounts of d4T were detected after incubation with the liver homogenate.

### Example 5

### Stability of d4T-5′-[-bromophenyl methoxyalaninyl phosphate] and Ala-d4T-MP in Gastric and Intestinal Fluids

Simulated gastric and intestinal fluids were prepared following United States Pharmacopia USPXXII methods and were spiked with d4T-5′-[p-bromophenyl methoxyalaninyl phosphate] and Ala-d4T-MP to yield a solution with a final concentration of 100 μM of each compound. The spiked fluids were then placed in a 37° C. water bath. At a predetermined time, 100 μl aliquots of the spiked gastric or intestinal fluid were extracted by adding 400 μl of acetone as discussed above.

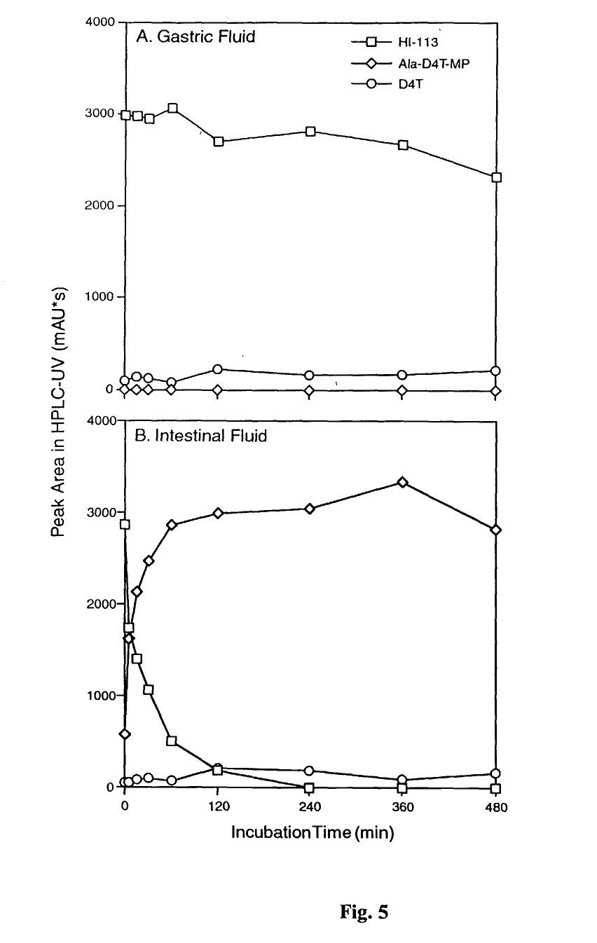

d4T-5′-[p-bromophenyl methoxyalaninyl phosphate] is relatively stable in gastric fluid for 8 hours, but it is not stable in intestinal fluid (FIGS. 5A and 5B). d4T-5′-[p-bromophenyl methoxyalaninyl phosphate] quickly decomposed to yield Ala-d4T-MP in intestinal fluid (approximately 94% of the d4T-5′-[p-bromophenyl methoxyalaninyl phosphate] had decomposed within 2 hours). Ala-d4T-MP was stable in intestinal fluid; only a trace amount of d4T was detected in the intestinal fluid.

### Example 6

### Pharmacokinetic Studies in Mice

Female Balb/c mice (6-8 weeks old) from Taconic (Germantown, N.Y.) were housed in a controlled environment (12-hours of light/12-hours of dark, 22±1° C., 60±10% relative humidity), which is fully accredited by the USDA. All rodents were housed in microisolator cages (Lab Products, Inc., NJ) containing autoclaved bedding. The mice were allowed free access to autoclaved pellet food and tap water throughout the study. All animal care procedures conformed to the Guide for the Care and Use of Laboratory Animals (National Research Council, National Academy Press, Washington DC 1996).

A solution (50 μl) of d4T-5 ′-[p-bromophenyl methoxyalaninyl phosphate] (100 mg/kg) dissolved in DMSO was administered intravenously via the tail vein. This volume of DMSO is well-tolerated by mice when administrated by rapid intravenous or extravascular injection (Rosenkrantz et al., Cancer Chemother. Rep., 31, 7 (1963); Wilson et al., Toxicol. Appl. Pharmacol., 7, 104 (1965)). Blood samples (˜500 μL) were obtained from the ocular venous plexus by retro-orbital venipuncture at 0, 2, 5, 10, 15, 30, 45, 60, 120, 240 and 360 minutes after intravenous injection. In order to study the pharmacokinetics of Ala-d4T-MP and d4T following systemic administration of these compounds, mice were injected with 75 mg/kg Ala-d4T-MP and 40 mg/kg d4T, respectively (these doses are equimolar to the 100 mg/kg d4T-5′-[p-bromophenyl methoxyalaninyl phosphate]).

In order to determine the pharmacokinetics of d4T-5′-[p-bromophenyl methoxyalaninyl phosphate] following oral administration, 12 hour fasted mice were given a bolus dose of 100 mg/kg d4T-5′-[p-bromophenyl methoxyalaninyl phosphate] via gavage using a #21 stainless-steel ball-tipped feeding needle. Sampling time points were 0, 2, 5, 10, 15, 30, 45, 60, 120, 240 and 360 minutes after the gavage.

All collected blood samples were heparinized and centrifuged at 7000×g for 5 minutes to separate the plasma fraction from the whole blood. The plasma samples were then processed immediately using the extraction procedure described above.

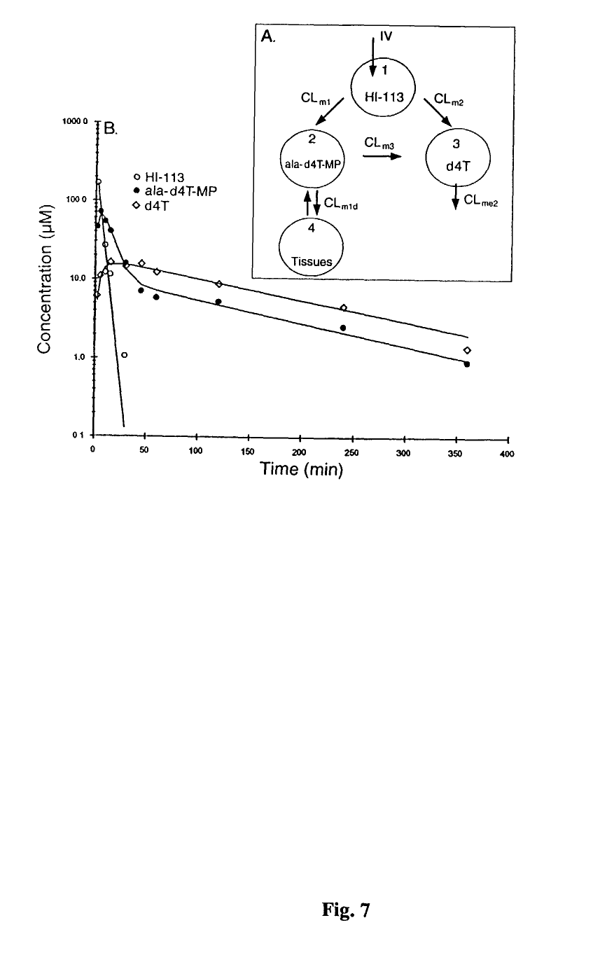

Pharmacokinetic modeling and parameter calculations were carried out using the WinNonlin Professional Version 3.0 (Pharsight, Inc., Mountain, Calif.) pharmacokinetics software (Chen et al., Pharm. Res., 16, 1003 (1999); Chen et al., Pharm. Res., 16, 117 (1999); Chen et al., J. Clin. Pharmacol., 39, 1248 (1999); Uckun et al., Clin. Cancer Res., 5, 2954 (1999); and Uckun et al., J. Pharmacol. Exp. Ther., 291, 1301 (1999)). An appropriate model was chosen on the basis of the lowest sum of weighted squared residuals, the lowest Schwartz Criterion (SC), the lowest Akaike's Information Criterion (AIC) value, the lowest standard errors of the fitted parameters, and the dispersion of the residuals. The elimination half-life was estimated by linear regression analysis of the terminal phase of the plasma concentration-time profile. The area under the concentration-time curve (AUC) was calculated according to the linear trapezoidal rule between the first sampling time (0 hours) and the last sampling time plus C/k, where C is the concentration of the last sampling and k is the elimination rate constant. The systemic clearance (CL) was determined by dividing the dose by the AUC. The metabolic clearance of the parent drug, the formation clearance of the metabolite, the clearance elimination of the metabolite, and the distribution clearance of the metabolite were estimated by simultaneous fitting of the concentration of parent drug and metabolites as a function of time curve to pharmacokinetic models (see FIGS. 7A & 8A) specified as a system of differential equations (Gabrielsson & Weiner, Phamacokinetic/Phamacodynamic Data Analysis: Concepts and Applications, Swedish Pharmaceutical Press (1997)). The fraction of d4T-5′-[p-bromophenyl methoxyalaninyl phosphate] converted to a metabolite (fₘ) was calculated as the ratio of the AUC for the metabolite after administration of the parent drug [(AUCₘ)ₚ] to the AUC after administration of an equimolar dose of the metabolite [(AUCₘ)ₘ] (Gibaldi & Perrier, 1982):

fₘ=[(AUCₘ)ₚ/Dₚ]×[Dₘ/(AUCₘ)ₘ]=(AUCₘ)ₚ·CLₘ/Dₚ

### Example 7

### Metabolism and Pharmacokinetic Profile of d4T-5′-[p-bromophenyl methoxyalaninyl phosphate] Following Intravenous Administration

Following intravenous administration, d4T-5′-[p-bromophenyl methoxyalaninyl phosphate] (100 mg/kg) was metabolized to yield d4T-5′-[p-bromophenyl methoxyalaninyl phosphate]-M1 (R_{T}=15.3 minutes) and d4T-5′-[p-bromophenyl methoxyalaninyl phosphate]-M2 (R_{T}=18.5 minutes) (FIG. 1C). d4T-5′-[p-bromophenyl methoxyalaninyl phosphate]-M1 had the same retention time as Ala-d4T-MP, whereas d4T-5′-[p-bromophenyl methoxyalaninyl phosphate]-M2 had the same retention time as d4T (FIGS. 1B and 1C). The UV spectra of these two metabolites were identical to those of Ala-d4T-MP and d4T, respectively.

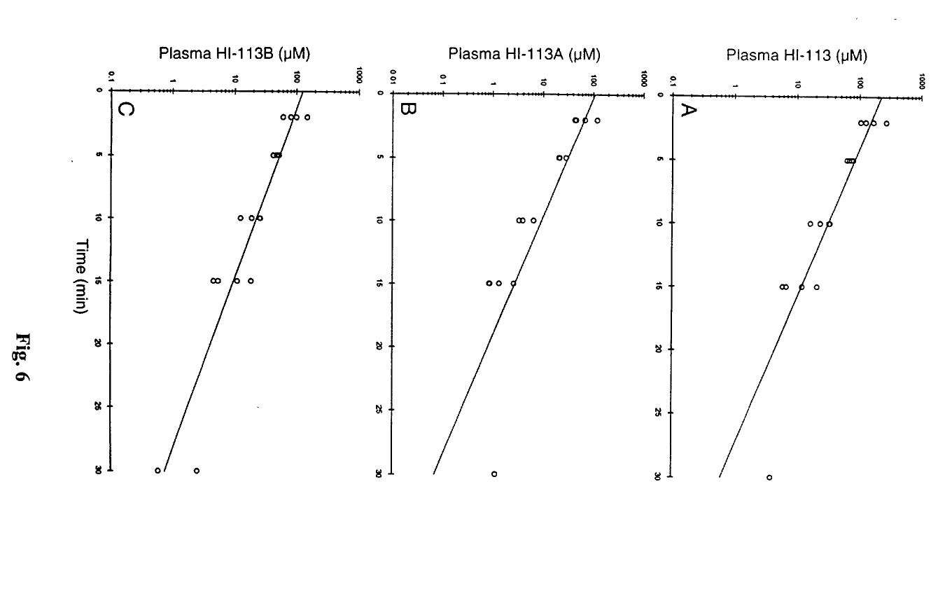

After intravenous administration of 100 mg/kg d4T-5 ′-[p-bromophenyl methoxyalaninyl phosphate], the plasma concentration of d4T-5′-[p-bromophenyl methoxyalaninyl phosphate] as a function of time was described by a one-compartment model (FIG. 6A). The estimated pharmacokinetic parameter values are presented in Table 2. d4T-5′-[p-bromophenyl methoxyalaninyl phosphate] had a Cₘₐₓ of 224.2 μM and an AUC of 1142.0 μM·minute. The systemic clearance of d4T-5′-[p-bromophenyl methoxyalaninyl phosphate] was moderately fast with a CL of 160.9 mL/minute/kg, which is approximately twice the rate of blood flow to the kidney or the liver (Davies et al., Pharm. Res., 10, 1093 (1993)). d4T-5′-[p-bromophenyl methoxyalaninyl phosphate] had a moderate size volume of distribution with a V_{SS} of 819.9 ml/kg, which is roughly equal to the total volume of water in the body. d4T-5′-[p-bromophenyl methoxyalaninyl phosphate] had a short elimination half-life (t_{1/2}=3.5 minutes), however, because of its rapid metabolism.

The diastereomers of d4T-5′-[p-bromophenyl methoxyalaninyl phosphate] were separated using the HPLC conditions described above (the retention times were 28.7 and 28.9 minutes). One of the diastereomers (d4T-5′-[p-bromophenyl methoxyalaninyl phosphate]-A, retention time=28.7 minutes) was metabolized more quickly than the other (d4T-5′-[p-bromophenyl methoxyalaninyl phosphate]-B, retention time=28.9 minutes; FIG. 1C). The pharmacokinetic features of these two diastereomers are summarized in Table 2. d4T-5′-[p-bromophenyl methoxyalaninyl phosphate]-B had a higher AUC (741.2 vs. 441.5 μM·minute) and Cₘₐₓ (125.7 vs. 107.9 μM) than d4T-5′-[p-bromophenyl methoxyalaninyl phosphate]-A (FIGS. 6B and 6C). d4T-5′-[p-bromophenyl methoxyalaninyl phosphate]-B also had a slightly longer elimination half-life than the d4T-5′-[p-bromophenyl methoxyalaninyl phosphate]-A diastereomer (4.1 minutes vs. 2.8 minutes), which may be due to faster clearance of d4T-5′-[p-bromophenyl methoxyalaninyl phosphate]-A relative to that of d4T-5′-[p-bromophenyl methoxyalaninyl phosphate]-B (208.2 vs. 123.9 ml/min/kg). However, both d4T-5′-[p-bromophenyl methoxyalaninyl phosphate] diastereomers were completely metabolized within 30 minutes.

Following intravenous injection, d4T-5′-[p-bromophenyl methoxyalaninyl phosphate] was rapidly metabolized to yield Ala-d4T-MP (tₘₐₓ=5.9 minutes; Cₘₐₓ=67.4 μM; t_{1/2}=129.2 minutes) and d4T (tₘₐₓ=18.7 minutes; Cₘₐₓ=15.7 μM; t_{1/2}=114.8 minutes) as shown in Table 2.

TABLE 2
Estimated Pharmacokinetic Parameter Values for d4T-5′-[p-bromophenyl
methoxyalaninyl phosphate] and Its Metabolites in Balb/C Mice

| | Vₛₛ | AUC | Cₘₐₓ | t_{1/2} | CL | tₘₐₓ |
|---|---|---|---|---|---|---|
| Measured | (ml/kg) | (μM · min) | (μM) | (min) | (ml/min/kg) | (min) |
| Total d4T-5′-[p- | 819.9 | 1142.0 | 224.2 | 3.5 | 160.9 | ND |
| bromophenyl | (920 ± 127.4) | (1071.8 ± 81.8) | (211.6 ± 29.3) | (3.6 ± 0.3) | (174.5 ± 13.2) | |
| methoxyalaninyl | | | | | | |
| phosphate] | | | | | | |
| d4T-5′-[p- | 852.1 | 441.5 | 107.9 | 2.8 | 208.2 | ND |
| bromophenyl | (1005.3 ± 134.0) | (359.7 ± 43.9) | (96.5 ± 12.8) | (2.6 ± 0.1) | (266.5 ± 30.2) | |
| methoxyalaninyl | | | | | | |
| phosphate]-A | | | | | | |
| d4T-5′-[p- | 731.1 | 741.2 | 125.7 | 4.1 | 123.9 | ND |
| bromophenyl | (791.8 ± 113.1) | (730.8 ± 45.7) | (123.1 ± 16.8) | (4.3 ± 0.4) | (127.3 ± 8.2) | |
| methoxyalaninyl | | | | | | |
| phosphate]-B | | | | | | |
| Ala-d4T-MP | ND | 2854.8 | 67.4 | 129.2 | ND | 5.9 |
| | | (2795.9 ± 361.2) | (69.3 ± 4.1) | (138.8 ± 40.2) | | (5.1 ± 0.7) |
| d4T | ND | 2915.2 | 15.7 | 114.8 | ND | 18.7 |
| | | (2858.1 ± 182.2) | (15.6 ± 1.2) | (116.2 ± 11.9) | | (17.4 ± 2.6) |

Pharmacokinetic parameters in Balb/c mice (N=4 mice per time-point) are presented as the average values estimated from composite plasma concentration-time curves of pooled data. The mean±S.E.M values are indicated in parentheses. ND means the value was not determined.

The model depicted in FIGS. 7A and 7B describes the metabolite pharmacokinetics of Ala-d4T-MP and d4T after intravenous injection of d4T-5′-[p-bromophenyl methoxyalaninyl phosphate]. According to this model, d4T-5′-[p-bromophenyl methoxyalaninyl phosphate] is biotransformed to produce Ala-d4T-MP (CLₘ₁) and d4T (CLₘ₂), respectively. Ala-d4T-MP derived from d4T-5′-[p-bromophenyl methoxyalaninyl phosphate] can be further metabolized to form D4T (CLₘ₃) or distributed to the extravascular compartment (Cl_{m1d}). D4T produced from either d4T-5′-[p-bromophenyl methoxyalaninyl phosphate] or Ala-d4T-MP is finally eliminated from the body (CLₘₑ₂). The pharmacokinetic parameters estimated for these two metabolites are presented in Table 3.

TABLE 3
Estimated Metabolite Pharmacokinetic Parameter Values

| Phamacokinetic | |
|---|---|
| Parameter | ml/min/kg |
| CLₘ₁ | 83.9 (21.5%) |
| CLₘ₂ | 87.4 (24.4%) |
| CLₘ₃ | 36.1 (85.9%) |
| CL_{mld} | 62.0 (69.8%) |
| CLₘₑ₂ | 47.1 (74.1%) |

The values in parenthesis are the C.V. of modeling. The metabolic clearance of d4T-5′-[p-bromophenyl methoxyalaninyl phosphate] and the formation clearance of the metabolites were 83.9 ml/minute/kg for Ala-d4T-MP and 87.4 ml/minute/kg for d4T, respectively. The metabolic clearance of Ala-d4T-MP and the formation clearance of its metabolite, d4T, were 36.1 ml/minute/kg, and a small portion of Ala-d4T-MP was distributed to the extravascular compartment with a CL_{m1d} of 47.1 ml/minute/kg. Finally, d4T was eliminated with a CLₘₑ₂ of 62.0 ml/minute/kg.

### Example 8

### Pharmacokinetic Profile of Ala-d4T-MP Following Intravenous Administration

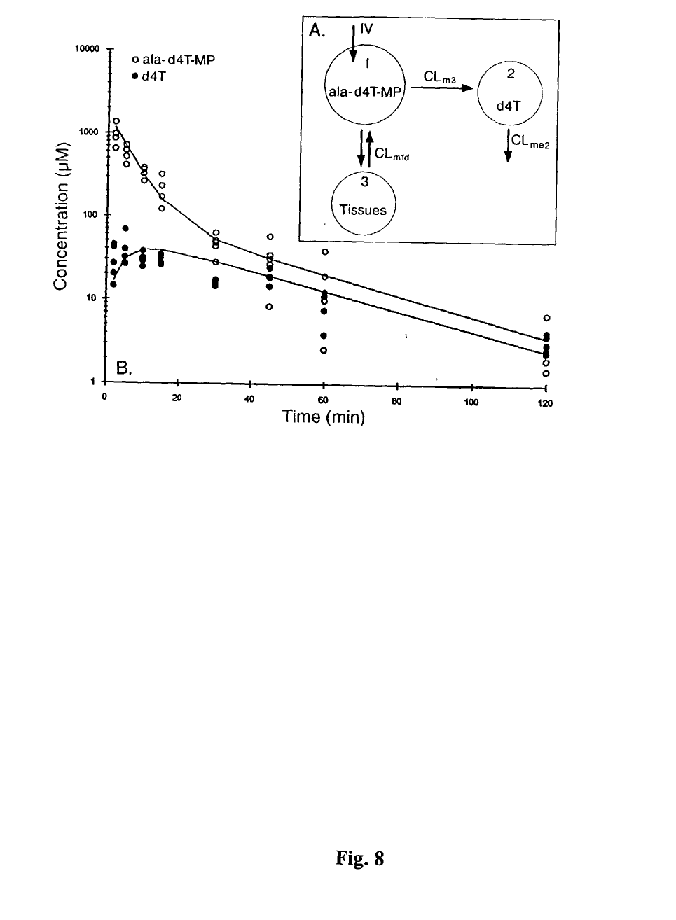

Following intravenous injection of Ala-d4T-MP (75 mg/kg, a dose equimolar to the 100 mg/kg dose of d4T-5′-[p-bromophenyl methoxyalaninyl phosphate] discussed above), Ala-d4T-MP was quickly metabolized to yield d4T (tₘₐₓ=4.4 minutes; t_{1/2}=34.0 minutes) (FIGS. 8A and 8B, Table 4). The concentration of Ala-d4T-MP as a function of time can be described using a two-compartment model, while a one-compartment model best fits the concentration of its metabolite, d4T, as a function of time (FIG. 8B). The Cₘₐₓ values for Ala-d4T-MP and d4T were 1206.6 μM and 35.2 μM, respectively. The AUC was 11648.7 μM·minute for Ala-d4T-MP and 1888.0 μM·minute for d4T. The systemic clearance of Ala-d4T-MP was only 15.8 mL/minute/kg (Table 4), which is much less than the blood flow to either the kidney or the liver (Davies et al., Pharm. Res., 10, 1093 (1993)). Ala-d4T-MP also had a small volume of distribution (V_{SS}=275.5 ml/kg) that is less than the total volume of water in the body. Nevertheless, the elimination half-life of Ala-d4T-MP was short (t_{1/2}=28.5 minutes), due to its rapid metabolism.

TABLE 4
Estimated Pharmacokinetic Parameter Values for Ala-d4T-MP
and Its Metabolite in Balb/C Mice

| | Vss | AUC | Cₘₐₓ | t_{1/2} | CL | tₘₐₓ |
|---|---|---|---|---|---|---|
| Measured | (ml/kg) | (μM · min) | (μM) | (min) | (ml/min/kg) | (min) |
| Ala-d4T- | 275.5 | 11648.7 | 1206.6 | 28.5 | 15.8 | ND |
| MP | (412.6 ± 126.3) | (11761.5 ± 447.2) | (1658.1 ± 544.9) | (91.5 ± 54.5) | (15.7 ± 0.6) | |
| d4T | ND | 1888.0 | 35.2 | 34.0 | ND | 4.4 |
| | | (1818.2 ± 42.9) | (35.9 ± 3.7) | (32.4 ± 2.2) | | (5.0 ± 1.2) |

Pharmacokinetic parameters in Balb/c mice (N=5 mice per time-point) are presented as the average values estimated from composite plasma concentration-time curves of pooled data. The mean±S.E.M values are indicated in parentheses. ND means the value was not determined.

The model depicted in FIG. 8A best described the metabolite pharmacokinetics after intravenous injection of Ala-d4T-MP. According to this model, Ala-d4T-MP can either be metabolized to form d4T (CLₘ₁) or distributed to the extravascular compartment (Cl_{pd}). D4T derived from Ala-d4T-MP is eliminated from the body (CLₘₑ). By simultaneous fitting of the parent Ala-d4T-MP and d4T concentration values as a function of time to the described model, the metabolic clearance of Ala-d4T-MP and the formation clearance of d4T (CLₘ₁) were estimated to be 15.6 ml/min/kg as shown in Table 5.

TABLE 5
Estimated Metabolite Pharmacokinetic Parameter Values

| Pharmacokinetic | |
|---|---|
| Parameter | ml/min/kg |
| CLₘₗ | 15.6 (16.6%) |
| CLₘₑ | 88.4 (13.0%) |
| CL_{pd} | 4.7 (44.7%) |

The data in parentheses are the C.V. of modeling. A small portion of Ala-d4T-MP was distributed to extravascular compartment with a CL_{pd} of 4.7 ml/minute/kg and d4T derived from Ala-d4T-MP was finally eliminated with a relatively high CLₘₑ of 88.4 ml/minute/kg. The CLₘ₁ of 15.6 ml/minute/kg accounts for 99% of the total systemic clearance (CL=15.8 ml/minute/kg) (see Table 4), indicating that most of Ala-d4T-MP was biotransformed to form d4T.

### Example 9

### Pharmacokinetic Profile of d4T Following Intravenous Administration

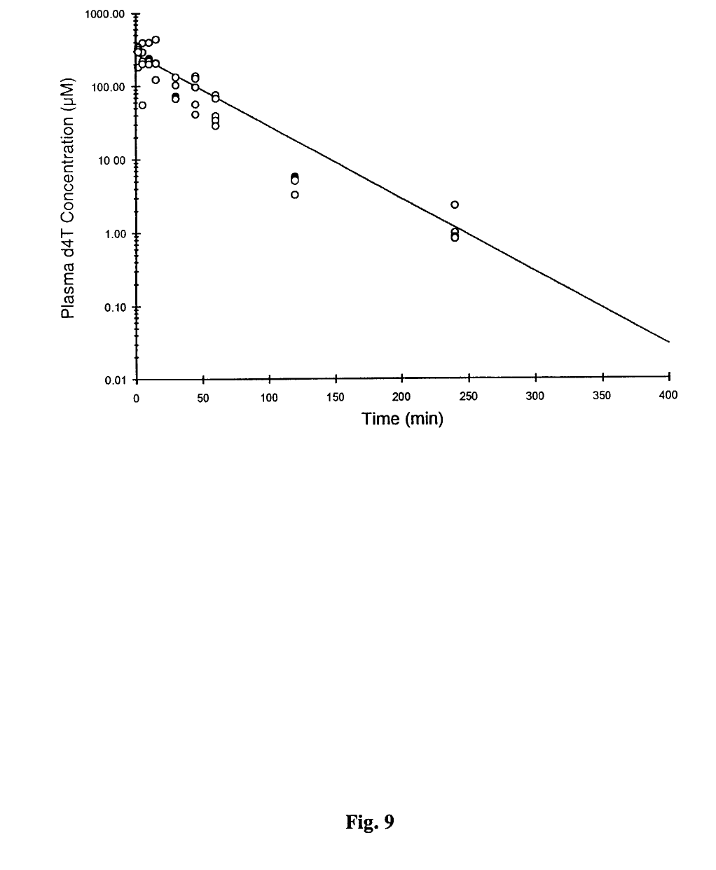

Following intravenous injection at a dose level of 40 mg/kg, a dose equimolar to the 100 mg/kg dose of d4T-5′-[p-bromophenyl methoxyalaninyl phosphate], the concentration of d4T as a function of time was described using a one-compartment model (FIG. 9). The estimated pharmacokinetic parameter values are presented in Table 6. The estimated Cₘₐₓ and AUC values for D4T were 279.5 μM and 12227.1 μM·minute, respectively. D4T had a short elimination half-life (30.3 minutes). The systemic clearance of d4T was slow with a CL of only 15.0 ml/min/kg, which is much lower than the blood flow to either the kidney or the liver (Davies et al., Pharm. Res., 10, 1093 (1993)). D4T had a moderately large volume of distribution (V_{SS}=657.8 ml/kg) that is approximately equal to the volume of water in the body.

TABLE 6
Estimated Pharmacokinetic Parameter Values for D4T in Balb/C Mice

| | Vss | AUC | Cₘₐₓ | t_{1/2} | CL |
|---|---|---|---|---|---|
| Measured | (ml/kg) | (μM · min) | (μM) | (min) | (ml/min/kg) |
| d4T | 657.8 | 12227.1 | 279.5 | 30.3 | 15.0 |
| | (581.8 ± 62.8) | (12173.6 ± 559.5) | (318.9 ± 15.7) | (26.6 ± 1.2) | (15.2 ± 0.7) |

Pharmacokinetic parameters in Balb/c mice (N=5 mice per time-point) are presented as the average values estimated from composite plasma concentration-time curves of pooled data. The mean±S.E.M values are indicated in parentheses.

### Example 10

### Pharmacokinetic Profile of d4T-5′-[p-bromophenyl methoxyalaninyl phosphate] Following Oral Administration

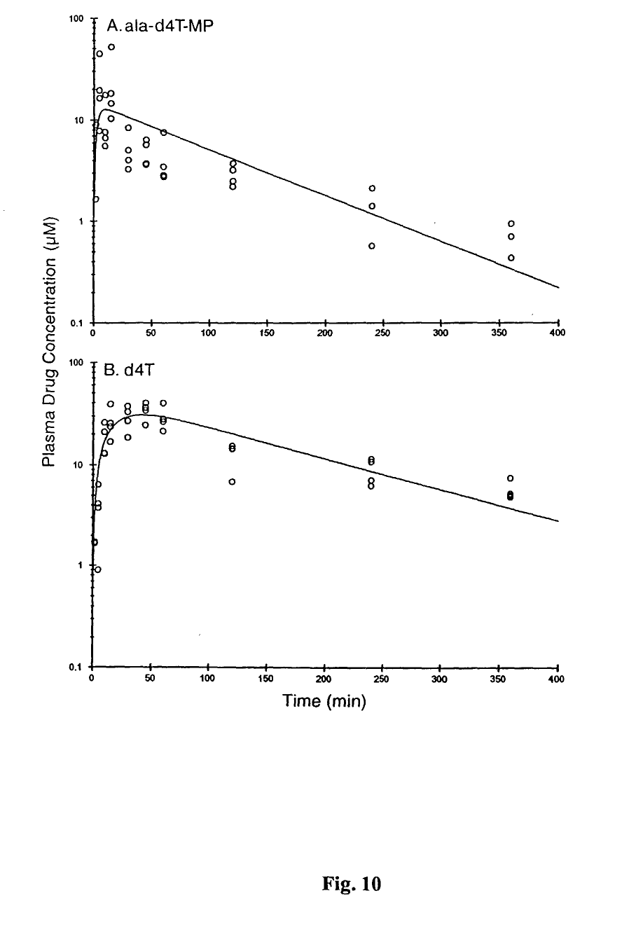

The pharmacokinetic behavior of orally administered d4T-5′-[p-bromophenyl methoxyalaninyl phosphate] (100 mg/kg) was also examined. Both metabolites (Ala-d4T-MP and d4T) were detected, but the concentration of the parent d4T-5′-[p-bromophenyl methoxyalaninyl phosphate] was below the detection limit (0.5 μM). The tₘₐₓ values are 10.3 minutes for Ala-d4T-MP and 42.4 minutes for d4T. A one-compartment pharmacokinetic model was used to describe both the Ala-d4T-MP and the d4T concentration changes as a function of time (FIGS. 10A and 10B). The estimated values for the pharmacokinetic parameters are presented in Table 7. The maximum concentrations (Cₘₐₓ) for Ala-d4T-MP and D4T are 12.7 μM and 30.7 μM, respectively. The elimination half-lives were 66.4 minutes and 99.0 minutes for Ala-d4T-MP and d4T, respectively.

TABLE 7
Estimated Pharmacokinetic Parameter Values
Following Oral Administratio of d4T-5′-[p-bromophenyl
methoxyalaninyl phosphate] in Balb/C Mice

| | AUC | Cₘₐₓ | t_{1/2} | tₘₐₓ |
|---|---|---|---|---|
| Measured | (μM · min) | (μM) | (min) | (min) |
| Ala-d4T- | 1350.5 | 12.7 | 66.4 | 10.3 |
| MP | (1355.4 ± 88.2) | (15.6 ± 4.1) | (56.1 ± 8.5) | (9.3 ± 0.9) |
| d4T | 5905.3 | 30.7 | 99.0 | 42.4 |
| | (5928.4 ± 294.6) | (29.5 ± 0.3) | (102.6 ± 3.8) | (45.2 ± 5.2) |

Pharmacokinetic parameters in Balb/c mice (N=4 mice per time-point) are presented as the average values estimated from composite plasma concentration-time curves of pooled data. The mean±S.E.M values are indicated in parentheses.

### Example 11

### Treatment of Lassa Virus Infected Mice with d4T-5′-[p-bromophenyl methoxyalaninyl phosphate]

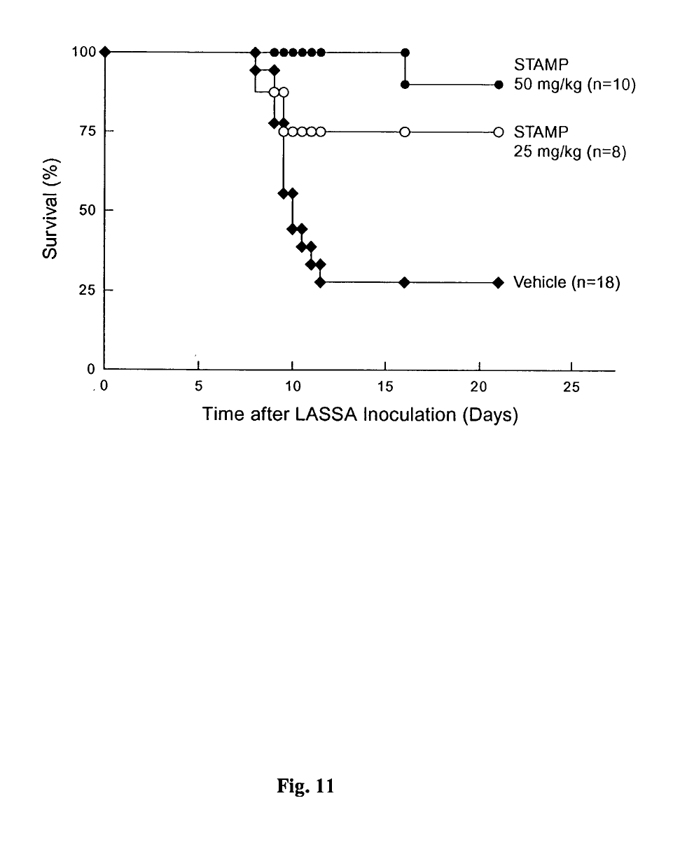

CBA mice were inoculated with intracerebral injections of the Josiach strain of Lassa Virus at a dose of 1000 PFU. This dose is known to be lethal to 70-100% of mice within 7-12 days (See, for example, Fidarov et al., Vopr Virusol 1990 Jul.-Aug.;35(4):326-9; and Ignat'ev et al., Vopr Virusol 1994 Nov.-Dec.;39(6):257-60). Mice were treated with vehicle (control) or with d4T-5′-[p-bromophenyl methoxyalaninyl phosphate] administered intraperitoneally 24 hours prior to, 1 hour prior to, and 24 hours, 48 hours, 72 hours, and 96 hours after virus inoculation. Mice were then observed twice daily for 21 days for morbidity and mortality. Of the 20 control mice, 2 died on day 1 immediately after intracerebral injection due to accidental brain injury and were not evaluable. All of the remaining 18 vehicle-treated control mice developed decreased mobility and scruffy fur as the clinical signs of Lassa infection between days 6 and 9 (Table 8). Sixteen of the 18 control mice developed seizures between days 7 and 11. Thirteen mice experienced 5-10% weight loss and died between days 8 and 11 (Table 8, FIG. 11). Of the 10 mice treated with d4T-5′-[p-bromophenyl methoxyalaninyl phosphate] at the 25 mg/kg dose level, two died accidentally immediately after intracerebral Lassa virus inoculation. All of the remaining 8 mice developed decreased mobility and scruffy fur as the clinical signs of Lassa infection between days 6 and 10. Two of these mice experienced 4-8% weight loss, developed seizures and died on days 8 and 10, respectively. The remaining 6 mice survived the Lassa challenge beyond the 21 day observation period and did not experience any weight loss or seizures (Table 8, FIG. 11). Of the 10 mice treated with d4T-5′-[p-bromophenyl methoxyalaninyl phosphate] at the 50 mg/kg dose level, only one mouse developed delayed signs of Lassa infection on day 13 as evidenced by decreased mobility and scruffy fur, lost weight, and died on day 16 after intracerebral Lassa virus inoculation. All 9 of the remaining mice remained healthy without clinical signs of Lassa infection beyond the 21-day observation period.

The probability of survival following the Lassa challenge was significantly improved for d4T-5′-[p-bromophenyl methoxyalaninyl phosphate] treated mice (Kaplan Meier, Chi-squared=11.7, df=2, Log-Rank p-value=0.003): The probability of survival at 21 days was 28% (7-48%, 95% confidence limits) for vehicle-treated mice (median survival=9 days), 75% (45-100%) for mice treated with d4T-5′-[p-bromophenyl methoxyalaninyl phosphate] at the 25 mg/kg dose level (median survival >21 days), and 90% (72-100%) for mice treated with d4T-5′-[p-bromophenyl methoxyalaninyl phosphate] at the 50 mg/kg dose level (median survival>21 days).

These results provide evidence that substituted aryl phosphate derivatives of d4T, such as d4T-5′-[p-bromophenyl methoxyalaninyl phosphate] are active anti-viral agents that inhibit the effects of HFV infection in animals, particularly Lassa virus infection. The data also demonstrates that these agents provide a prophylactic effect against HFV.

TABLE 8
Anti-LASSA Activity of d4T-5′-[p-bromophenyl
methoxyalaninyl phosphate] in CBA Mice

| Disease Onset: Days after | | | | |
|---|---|---|---|---|
| Inoculation with Lassa Virus | | | | |
| Decreased | Scruffy | | Weight | Survival |
| Mobility | Fur | Convulsions | Loss (%) | (days) |

Group A: Vehicle

| | | | | | |
|---|---|---|---|---|---|
| Mouse #1* | NA | NA | NA | NA | ≦1 |
| Mouse #2* | NA | NA | NA | NA | ≦1 |
| Mouse #3 | 6.0 | 6.0 | 7.0 | 4.5 | 8 |
| Mouse #4 | 6.0 | 6.5 | 8.0 | 5.0 | 9 |
| Mouse #5 | 7.5 | 7.0 | 8.0 | 9.0 | 9.5 |
| Mouse #6 | 7.5 | 7.5 | 8.0 | 4.3 | 9.0 |
| Mouse #7 | 7.0 | 7.0 | 8.0 | 4.3 | 9.0 |
| Mouse #8 | 7.0 | 7.0 | 8.5 | 4.5 | 9.5 |
| Mouse #9 | 7.0 | 7.0 | 8.5 | 8.3 | 9.5 |
| Mouse #10 | 7.0 | 7.0 | 8.5 | 4.8 | 9.5 |
| Mouse #11 | 8.5 | 8.0 | 9.0 | 5.3 | 10.5 |
| Mouse #12 | 8.5 | 8.0 | 9.0 | 4.5 | 10.0 |
| Mouse #13 | 8.0 | 8.5 | 9.5 | 4.5 | 10.0 |
| Mouse #14 | 9.0 | 9.0 | 10.5 | 4.1 | 11.0 |
| Mouse #15 | 9.0 | 9.5 | 11.0 | 4.3 | 11.5 |
| Mouse #16 | 9.0 | 10.0 | NO | NO | >21 |
| Mouse #17 | 9.0 | 10.0 | NO | NO | >21 |
| Mouse #18 | 9.0 | 10.0 | 11.0 | NO | >21 |
| Mouse #19 | 9.0 | 10.0 | NO | NO | >21 |
| Mouse #20 | 9.5 | 10.0 | NO | NO | >21 |

Group B - d4T-5′-[p-bromophenyl
methoxyalaninyl phosphate] 25 mg/kg

| | | | | | |
|---|---|---|---|---|---|
| Mouse #1* | NA | NA | NA | NA | ≦1 |
| Mouse #2* | NA | NA | NA | NA | ≦1 |
| Mouse #3 | 6.0 | 6.0 | 7.5 | 4.3 | 8 |
| Mouse #4 | 9.0 | 9.5 | 10.0 | 8.3 | 9.5 |
| Mouse #5 | 9.5 | 10.0 | NO | NO | >21 |
| Mouse #6 | 9.5 | 10.0 | NO | NO | >21 |
| Mouse #7 | 10.0 | 9.5 | NO | NO | >21 |
| Mouse #8 | 9.0 | 10.0 | NO | NO | >21 |
| Mouse #9 | 10.0 | 10.0 | NO | NO | >21 |
| Mouse #10 | 8.5 | 9.5 | NO | NO | >21 |

Group C - d4T-5′-[p-bromophenyl
methoxyalaninyl phosphate] 50 mg/kg

| | | | | | |
|---|---|---|---|---|---|
| Mouse #1 | 13 | 13 | NO | 8.7 | 16 |
| Mouse #2 | 9.0 | NO | NO | NO | >21 |
| Mouse #3 | 9.5 | 10.0 | NO | NO | >21 |
| Mouse #4 | NO | NO | NO | NO | >21 |
| Mouse #5 | NO | NO | NO | NO | >21 |
| Mouse #6 | NO | NO | NO | NO | >21 |
| Mouse #7 | NO | NO | NO | NO | >21 |
| Mouse #8 | NO | NO | NO | NO | >21 |
| Mouse #9 | NO | NO | NO | NO | >21 |
| Mouse #10 | NO | NO | NO | NO | >21 |

*Mouse died after traumatic intracerebral injection;
NA = not applicable;
NO = not observed

While a detailed description of the present invention has been provided above, the invention is not limited thereto. The invention described herein can be modified to include alternative embodiments, as will be apparent to those skilled in the art. All such alternatives should be considered within the spirit and scope of the invention, as claimed below.

The specification includes numerous citations to literature and patent references, each which is hereby incorporated by reference as if fully set forth, for all purposes.

We claim:

1. A method for inhibiting the effects of infection by hemorrhagic fever virus (HFV) in a cell, in vitro or in vivo, comprising administering to the cell an effective inhibitory amount of a compound of Formula I: 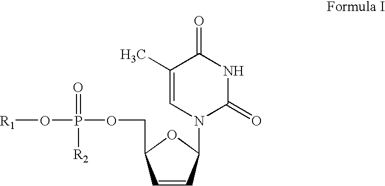
where R₁ is an aryl group substituted with an electron-withdrawing group or H, and
R₂ is an amino acid residue or an ester of the amino acid residue,
or a pharmaceutically acceptable salt thereof.

2. The method of claim 1, where R₁ is an aryl group substituted with an electron-withdrawing group.

3. The method of claim 2, wherein the aryl group is phenyl, naphthyl, or anthryl.

4. The method of claim 2, wherein the aryl group is phenyl.

5. The method of claim 2, wherein the electron-withdrawing group is a halo.

6. The method of claim 2, wherein R₁ is para-bromophenyl.

7. The method of claim 2, wherein R₁ is para-chlorophenyl.

8. The method of claim 1, wherein R₂ is an a-amino acid or ester thereof.

9. The method of claim 1, wherein R₂ is —NHCH(CH₃)COOCH₃.

10. The method of claim 2, wherein R₁ is para-bromophenyl and R₂ is —NHCH(CH₃)COOCH₃.

11. The method of claim 2, wherein R₁ is para-chlorophenyl and R₂ is —NHCH(CH₃)COOCH₃.

12. The method of claim 1, wherein the hemorrhagic fever virus is an arenavirus.

13. The method of claim 1, wherein the hemorrhagic fever virus is Lassa Virus.

14. The method of claim 1, wherein the administered compound is a compound of Formula IV: 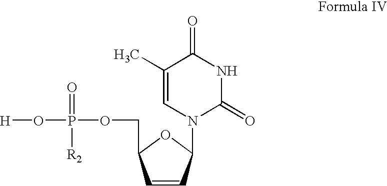
where R₂ is an amino acid residue or an ester of the amino acid residue, or a pharmaceutically acceptable salt thereof.

15. The method of claim 1, wherein said administering comprises administering to an animal.

16. The method of claim 15, wherein said compound is administered at a dose of about 1 mg/kg body weight to about 500 mg/kg body weight.

17. The method of claim 16, wherein said compound is administered at a dose of about 10 mg/kg body weight to about 100 mg/kg body weight.

18. The method of claim 15, wherein said inhibiting comprises reducing one or more symptom of HFV infection.

19. The method of claim 15, wherein said inhibiting comprises preventing or delaying the onset of one or more symptom of HFV infection.
